(19) United States
(12) Plant Patent Application Publication
(10) Pub. No.: US 2016/0205846 P1
(43) Pub. Date: Jul. 14, 2016

(54) Catalpa bungei 'Menghuan' plant named 'Colorful Manchurian Catalpa'

(50) ;

(71) Applicant: Henan Mingpin Colorized Seedling Co., Ltd., Suiping (CN)

(72) Inventor: Huaming Wang, Suiping (CN)

(21) Appl. No.: 14/999,003

(22) Filed: Mar. 16, 2016

(51) Int. Cl. A01H 5/00

(52) U.S. Cl. PLT/216

(57) ABSTRACT

‘Colorful Manchurian Catalpa’ is a distinctive variety of Catalpa Linn. Plant, which is characterized by that apical leaf edges have irregular rose-red areas, tender leaf edges have irregular yellow areas, and mature leaf edges have irregular white areas. The Colorful Manchurian Catalpa was discovered by the applicant according to: 1) On May 5, 2011, a mutant branch was found on a perennial Manchurian Catalpa at a road side in Long'an District, Anyang City, Henan Province, China. 2) On May 10, 2011, the mutant branch was bud-grafted onto an underyearling Catalpa rootstock with xylem kept, wherein it was observed that a new grafted plate was consistent with the mother plant in characteristics, and no changes happened. 3) From 2012 to 2014, mixed branches from varied generations were used for multiplying; according to observation, no nursery stock mutated, growth was strong, characteristics were kept, and performances were sufficient.

### LATIN NAME

Catalpa bungei ‘Menghuan’

### VARIETY DENOMINATION

Colorful Manchurian Catalpa

### BACKGROUND OF THE INVENTION

The present invention relates to a new and distinct variety of Catalpa bungei ‘Menghuan’ plant, which is given the varietal denomination of ‘Colorful Manchurian Catalpa’.

The new Colorful Manchurian Catalpa was discovered by the applicant according to:

1) On May 5, 2011, a mutant branch was found on a perennial Manchurian Catalpa at a road side in Long'an District, Anyang City, Henan Province, China; wherein after observation, it was found that leaf edge colors of apical leaves, tender leaves and mature leaves continuously changed from red, yellow to white.

2) On May 10, 2011, the mutant branch was bud-grafted onto an underyearling Catalpa rootstock with xylem kept, wherein it was observed that a new grafted plate was consistent with the mother plant in characteristics, and no changes happened.

3) From 2012 to 2014, mixed branches from varied generations were used for multiplying; according to observation, no nursery stock mutated, growth was strong, characteristics were kept, and performances were sufficient.

Conventionally, there are 3 first-generation plants, 22 second-generation plants and 961 third-generation plants, totally 986 plants.

### SUMMARY OF THE NEW VARIETY

‘Colorful Manchurian Catalpa’ is a distinctive variety of Catalpa bungei ‘Menghuan’ plant, which is a bud mutation of a Manchurian Catalpa. The Colorful Manchurian Catalpa is a deciduous arbor with a plurality of holes on a trunk thereof. Three leaves form a whorl, wherein the leaves are broadly ovate and lobed, with lengths of 6-15cm and widths of 8cm. Tops of the leaves are long and acuminate, and bases are truncate. Occasionally, there are 1-2 teeth on the bases. There is no hair on upper surfaces or lower surfaces of the leaves. Petioles are 2-8cm long. Axillarises at the lower surfaces of the leaves have a lot of plaques. Leaf edges are wavy. Apical leaf edges have irregular rose-red areas, tender leaf edges have irregular yellow areas, and mature leaf edges have irregular white areas. The trunk is straight, bark is gray, and tender branch bark is smooth with distinctive holes. As a contrast, leaf colors of an Asaka Catalpa are all green.

### BRIEF DESCRIPTION OF THE DRAWINGS

FIG. 1 is a color photograph of the new invention, ‘Colorful Manchurian Catalpa’, showing a whole plant.

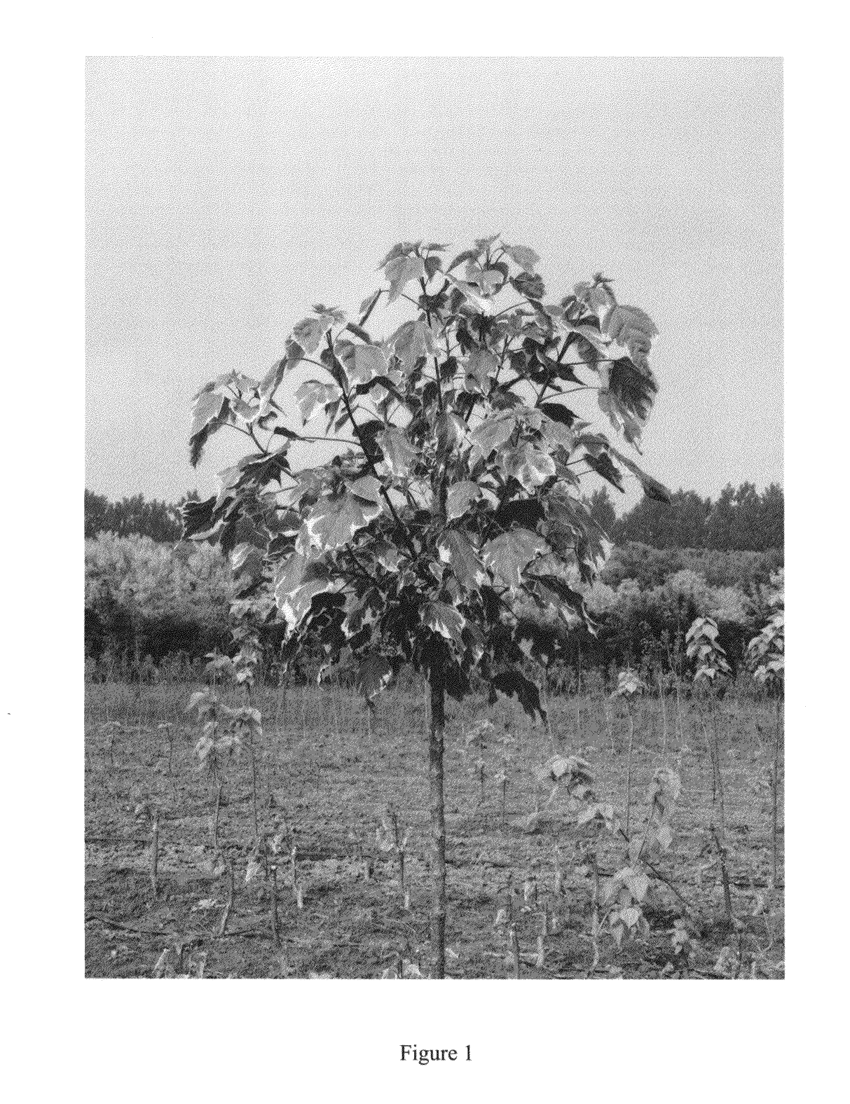

FIG. 2 is a color photograph of the new invention, ‘Colorful Manchurian Catalpa’, showing leaf shapes.

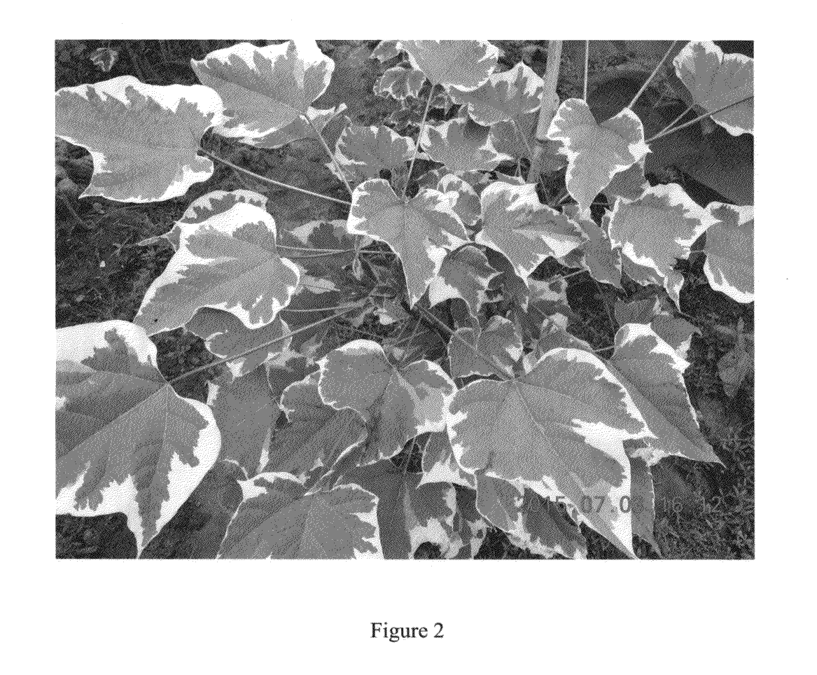

FIG. 3 is a color photograph of the new invention, ‘Colorful Manchurian Catalpa’, showing spring leaves.

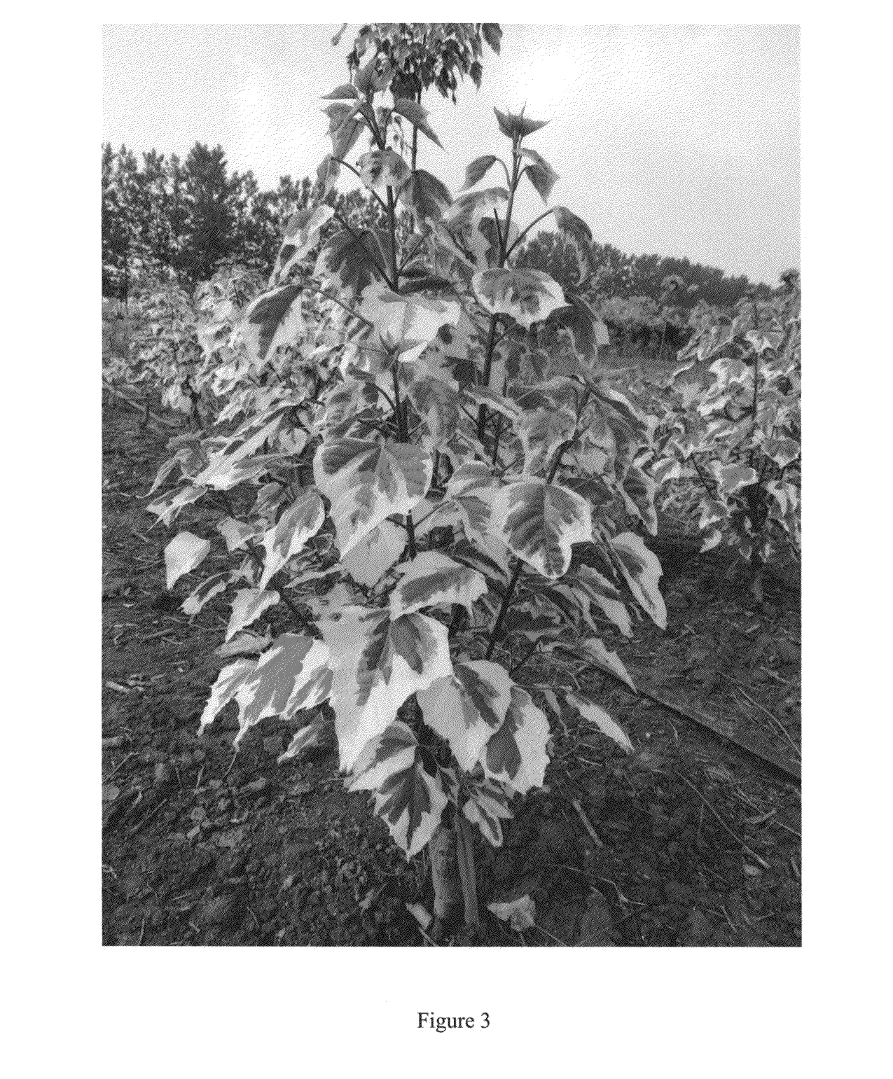

FIG. 4 is a color photograph of the new invention, ‘Colorful Manchurian Catalpa’, showing spring apical leaves.

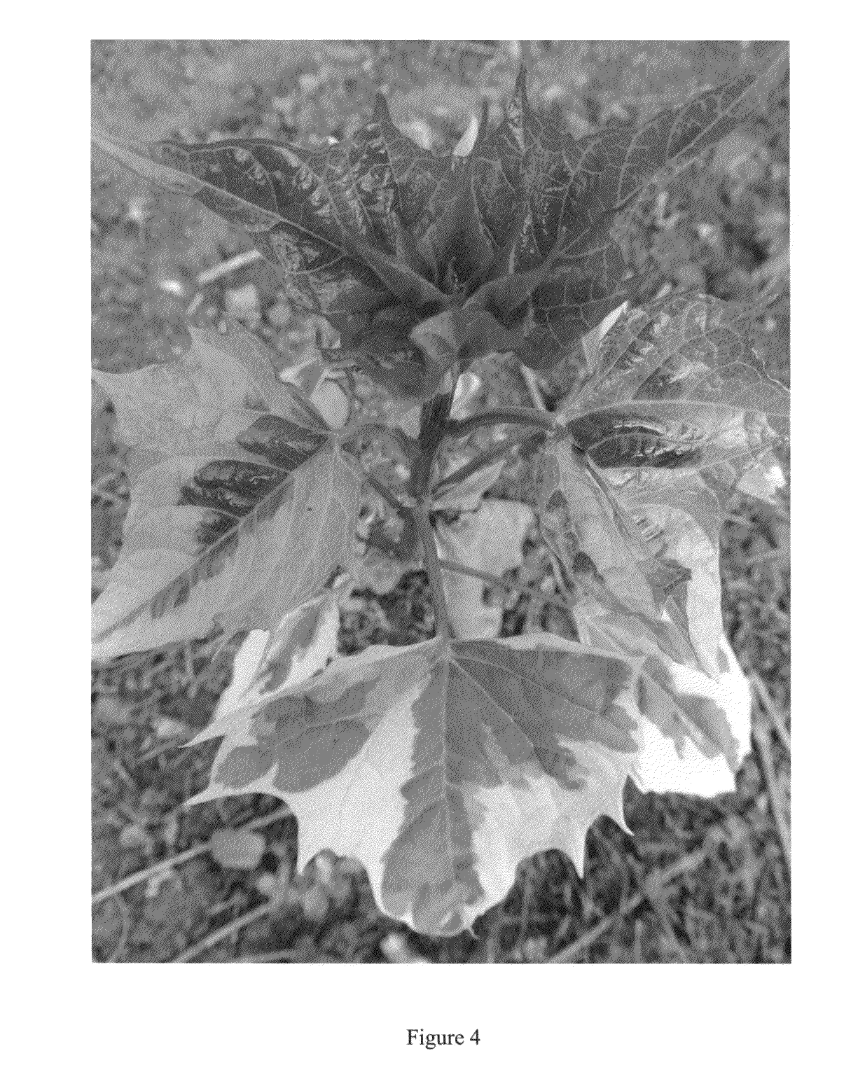

FIG. 5 is a color photograph of the new invention, ‘Colorful Manchurian Catalpa’, showing summer leaves.

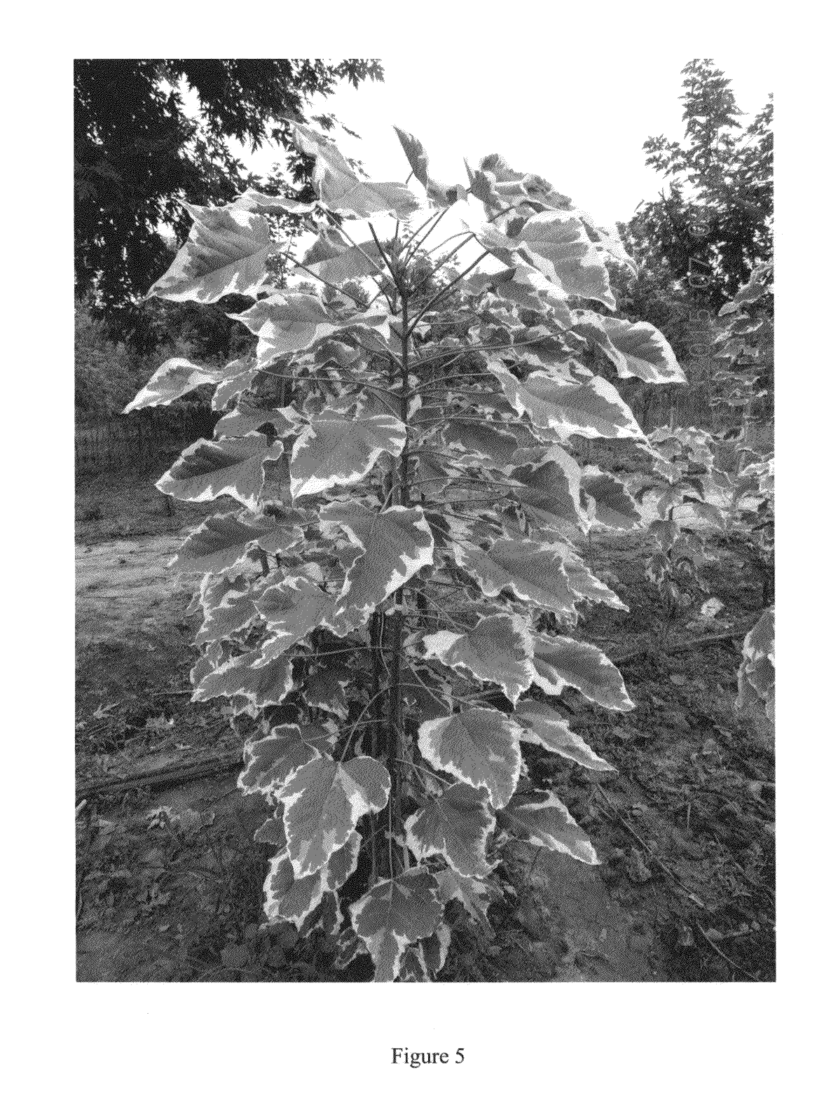

FIG. 6 is a color photograph of the new invention, ‘Colorful Manchurian Catalpa’, showing summer apical leaves.

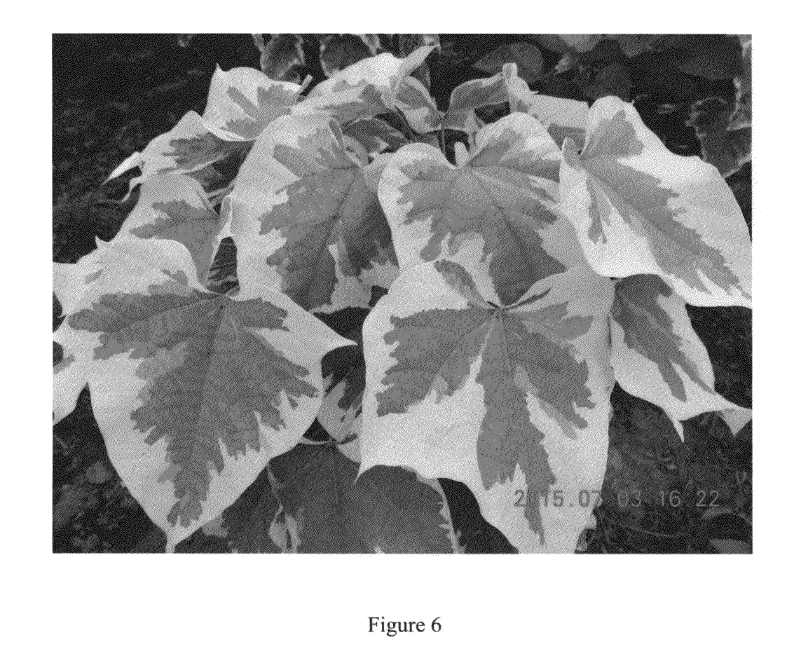

### DETAILED DESCRIPTION

The accompanying colored photographs illustrate the overall appearance of the new and distinct variety of the Catalpa bungei ‘Menghuan’ plant, which is known as ‘Colorful Manchurian Catalpa’. The descriptions disclosed herein are based upon observations of the plant growth.

The Colorful Manchurian Catalpa is a deciduous arbor. A young trunk has smooth bark. Apical leaf edges have irregular rose-red areas, tender leaf edges have irregular yellow areas, and mature leaf edges have irregular white areas.

| | feature | | |
|---|---|---|---|
| | leaf color | | |
| name | apical leaf | tender leaf | nature |
| Colorful | irregular rose-red | irregular yellow | irregular white area |
| Manchurian | area at edge | area at edge | at edge |
| Catalpa | | | |
| Asaka Catalpa | green | green | green |

By years of asexual reproduction, the Colorful Manchurian Catalpa is consistent and stable.

The Colorful Manchurian Catalpa demands light and is relatively cold-resistant.

Deep, fertile and moist soil is preferred, while the Colorful Manchurian Catalpa is drought-resistant, and slightly salinity-resistant. A tillers effect is strong, and lateral roots are well developed. There are strong anti-dust and anti-harmful gas capacities, and a life is long. The Colorful Manchurian Catalpa is able to grow at most areas of China due to a strong soil adaptability. However, well-drained, fertile and moist soil is preferred.

These features and other characteristics of the plant are apparent from the figures.

What is claimed is:

1. A new and distinct variety of plant named ‘Colorful Manchurian Catalpa’, substantially as described and illustrated, classified in the family Catalpa Linn., and characterized by that apical leaf edges have irregular rose-red areas, tender leaf edges have irregular yellow areas, and mature leaf edges have irregular white areas.
